(19) United States
(12) Patent Application Publication
(10) Pub. No.: US 2022/0321801 A1
(43) Pub. Date: Oct. 6, 2022

(54) DISPLAY CONTROL DEVICE

(71) Applicant: CANON MEDICAL SYSTEMS CORPORATION, Tochigi (JP)

(72) Inventor: Keisuke OKUZUMI, Utsunomiya (JP)

(73) Assignee: CANON MEDICAL SYSTEMS CORPORATION, Tochigi (JP)

(21) Appl. No.: 17/657,457

(22) Filed: Mar. 31, 2022

(30) Foreign Application Priority Data
Apr. 2, 2021 (JP) 2021-063698

(51) Int. Cl. H04N 5/268; H04N 7/18; G06V 40/10

(52) U.S. Cl. CPC H04N 5/268; H04N 7/181; G06V 40/10; H04N 2005/2255

(57) ABSTRACT

A display control device according to an embodiment includes a processing circuit. The processing circuit is configured to acquire a first image shot by a first camera capable of shooting an image of a subject outside a bore and a second image shot by a second camera capable of shooting an image of the subject inside the bore, and to display the first image and the second image on a display.

### CROSS-REFERENCE TO RELATED APPLICATIONS

This application is based upon and claims the benefit of priority from Japanese Patent Application No. 2021-063698, filed on Apr. 2, 2021; the entire contents of which are incorporated herein by reference.

### FIELD

Embodiments described herein relate generally to a display control device.

### BACKGROUND

In a medical imaging device such as a magnetic resonance imaging (MRI) device, it is desirable to automate patient settings for a subject.

It is possible to facilitate the automation of the patient setting by installing a camera on the ceiling of an imaging room, shooting an image of the subject from a position above the subject, and acquiring information about the subject, for example.

In addition, once the subject is moved to the imaging area, it is also desirable to acquire an image of the subject inside of the bore, as well as the image shot from above.

### BRIEF DESCRIPTION OF THE DRAWINGS

FIG. 1 is a schematic illustrating an example of a magnetic resonance imaging system in which a display control device according to an embodiment is included;

FIG. 2 is a schematic illustrating an example of the display control device according to the embodiment;

FIG. 3 is a drawing for explaining a process performed by the display control device according to a first embodiment;

FIG. 4 is a drawing for explaining a process performed by the display control device according to a second embodiment;

FIG. 5 is a drawing for explaining a process performed by the display control device according to the second embodiment;

FIG. 6 is a drawing for explaining a process performed by the display control device according to a third embodiment; and

FIG. 7 is a drawing for explaining a process performed by the display control device according to a third embodiment.

### DETAILED DESCRIPTION

A display control device provided according to one aspect of the present invention includes a processing circuit. The processing circuit is configured to acquire a first image shot by a first camera capable of shooting an image of a subject outside a bore and a second image shot by a second camera capable of shooting an image of the subject inside the bore, and to display the first image and the second image on a display.

Some embodiments of the display control device will now be explained in detail with reference to the drawings.

### First Embodiment

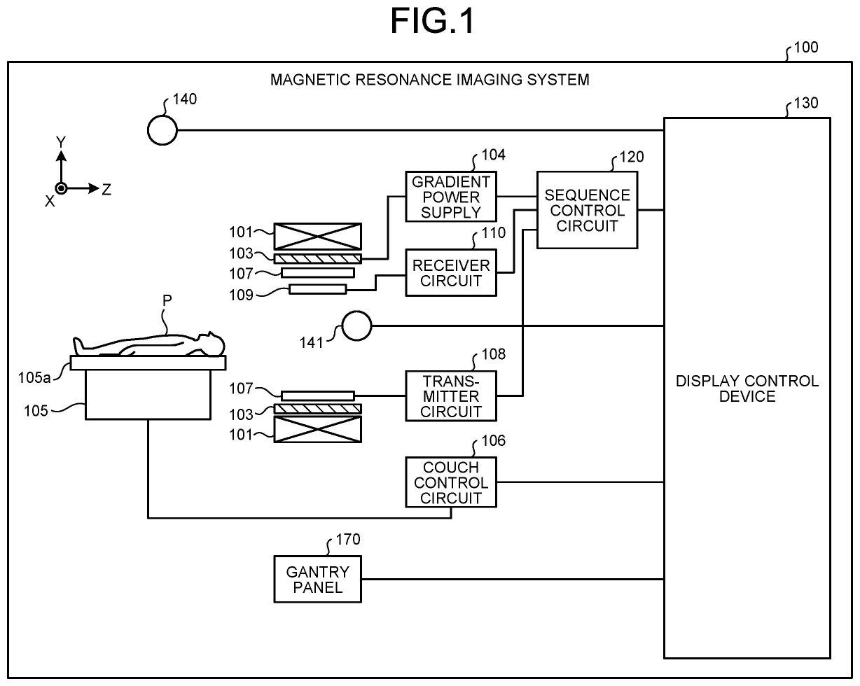

FIG. 1 is a schematic illustrating a configuration in which a display control device 130 according to an embodiment is included in a magnetic resonance imaging system 100. However, the embodiment is not limited to the example in which the display control device 130 is included in the magnetic resonance imaging system 100, and the display control device 130 may also be separate from the magnetic resonance imaging system 100. The display control device 130 may also be included in a system using a modality other than that of the magnetic resonance imaging system 100, e.g., an ultrasound diagnosis device.

As illustrated in FIG. 1, the magnetic resonance imaging system 100 includes a static field magnet 101, a static field power supply (not illustrated), a gradient coil 103, a gradient power supply 104, a couch 105, a couch control circuit 106, a transmitter coil 107, a transmitter circuit 108, a receiver coil 109, a receiver circuit 110, a sequence control circuit 120 (sequence controller), the display control device 130, a first camera 140, a second camera 141, and a gantry panel 170. A subject P (e.g., a human body) is not included in the magnetic resonance imaging system 100. The configuration illustrated in FIG. 1 is merely one example. For example, the components included in the sequence control circuit 120 and the display control device 130 may be integrated or separated as appropriate.

The static field magnet 101 is a magnet having a shape of a substantially hollow cylinder, and generates a static field in the direction along its central axis (Z-axis), in the space inside the cylinder. The static field magnet 101 is, for example, a superconducting magnet, and is excited by receiving a current supply from the static field power source. The static field power source supplies a current to the static field magnet 101. As another example, the static field magnet 101 may be a permanent magnet, and in such a configuration, the magnetic resonance imaging system 100 does not need to include a static field power source. The static field power source may also be provided separately from the magnetic resonance imaging system 100.

The gradient coil 103 is a substantially hollow, cylindrical coil, and is disposed on the inner side from the static field magnet 101. The gradient coil 103 is provided as a combination of three coils corresponding to X, Y, and Z axes, respectively, that are orthogonal to one another, and each of these three coils receives an individual current supply from the gradient power supply 104 and generates a gradient field in each of the X, Y, and Z axes directions, the intensity of the magnetic field in the Z direction changing on the basis of the distance from the center of each axis. The gradient fields along the X, Y, and Z axes generated by the gradient coil 103 are, for example, a gradient field Gs for slice encoding, a gradient field Ge for phase encoding, and a gradient field Gr for readout, respectively. The gradient power supply 104 supplies a current to the gradient coil 103.

The couch 105 has a couchtop 105a on which the subject P is laid, and inserts the couchtop 105a into the cavity (imaging port) of the gradient coil 103, with the subject P laid thereon, under the control of the couch control circuit 106. Usually, the couch 105 is installed in such a manner that its longitudinal direction extends in parallel with the central axis of the static field magnet 101. The couch control circuit 106 drives the couch 105 to move the couchtop 105a in longitudinal and vertical directions under the control of the display control device 130.

The transmitter coil 107 is disposed on the inner side from the gradient coil 103, and receives a radio frequency (RF) pulse from the transmitter circuit 108 and generates a high-frequency magnetic field. The transmitter circuit 108 supplies an RF pulse corresponding to the Larmor frequency that is determined by the type of the target atom and the magnetic field intensity, to the transmitter coil 107.

The receiver coil 109 is disposed on the inner side from the gradient coil 103, and receives a magnetic resonance signal (hereinafter referred to as an “MR signal” as required) emitted from the subject P by being affected by the high-frequency magnetic field. When the receiver coil 109 receives a magnetic resonance signal, the receiver coil 109 outputs the received magnetic resonance signal to the receiver circuit 110.

The transmitter coil 107 and the receiver coil 109 described above are merely one example. The transmitter coil 107 and the receiver coil 109 may be configured by one of or combining more than one of a coil only having a transmitting function, a coil only having a receiving function, and a coil having a transmitting function and a receiving function.

The receiver circuit 110 detects the magnetic resonance signal output from the receiver coil 109, and generates magnetic resonance data based on the detected magnetic resonance signal. Specifically, the receiver circuit 110 generates magnetic resonance data by performing a digital conversion on the magnetic resonance signal output from the receiver coil 109. The receiver circuit 110 also transmits the generated magnetic resonance data to the sequence control circuit 120. The receiver circuit 110 may also be provided to a gantry device where the static field magnet 101, the gradient coil 103, and the like are provided.

The sequence control circuit 120 captures an image of the subject P by driving the gradient power supply 104, the transmitter circuit 108, and the receiver circuit 110 based on sequence information received from the display control device 130. The sequence information herein is information that defines the sequence in which an image is captured. The sequence information defines, for example, the intensity of the current supplied by the gradient power supply 104 to the gradient coil 103 and the timing at which the current is supplied, the intensity of the RF pulse supplied by the transmitter circuit 108 to the transmitter coil 107 and the timing at which the RF pulse is applied, and the timing at which the receiver circuit 110 detects the magnetic resonance signal. The sequence control circuit 120 is, for example, an integrated circuit such as an application specific integrated circuit (ASIC) and a field programmable gate array (FPGA), and an electronic circuit such as a central processing unit (CPU) and a micro-processing unit (MPU).

The sequence control circuit 120 also transfers, upon receiving magnetic resonance data from the receiver circuit 110 as a result of driving the gradient power supply 104, the transmitter circuit 108, and the receiver circuit 110, and capturing an image of the subject P, the received magnetic resonance data to the display control device 130.

The first camera 140 is a camera capable of shooting an image of the subject P outside the bore, and is installed on the ceiling of the imaging room, for example. In this example, the first camera 140 shoots an image of the subject P from above.

The second camera 141 is a camera capable of shooting an image of the subject P inside the bore, and is installed on a side wall in the imaging room, for example.

The gantry panel 170 is a display panel provided to the gantry, and is a display device such as a liquid crystal display, for example. The gantry panel includes, for example, an input device and a display, and has the same functions as those of an input device 134 and of a display 135 included in the display control device 130.

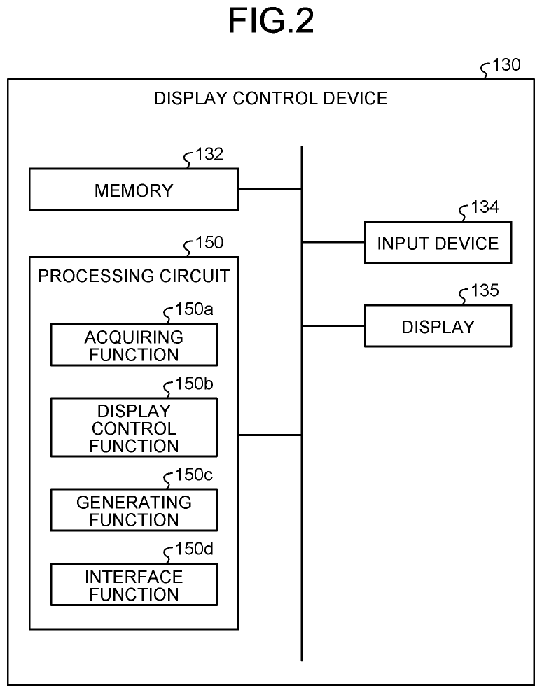

The display control device 130 not only controls to display an image having been acquired, but also performs processes such as controlling the overall magnetic resonance imaging system 100 and generating an image. FIG. 2 illustrates an example of a configuration of the display control device 130. The display control device 130 includes a memory 132, the input device 134, the display 135, and a processing circuit 150. The processing circuit 150 has an acquisition function 150a, a display control function 150b, a generating function 150c and an interface function 150d.

Explained below is an example in which the display control device 130 included in the magnetic resonance imaging system 100 functions as a display control device, but the embodiment is not limited to such an example. The display control device 130 may be independent from the magnetic resonance imaging system 100, and configured as a stand-alone display control device. In such a configuration, the processing circuit 150 included in the display control device 130 may be configured not to include some of the functions, e.g., a generating function 150c and an interface function 150d, with which the operation of the magnetic resonance imaging system 100 is controlled.

In the embodiment, each of the processing functions executed in an acquiring function 150a, a display control function 150b, the generating function 150c, and the interface function 150d is stored in the memory 132 as a computer program that is executable by a computer. The processing circuit 150 is a processor that implements the functions corresponding to the computer programs by reading the computer programs from the memory 132 and executing the computer programs. In other words, the processing circuit 150 having read the computer programs comes to have the functions illustrated in the processing circuit 150 in FIG. 2. In explaining FIG. 2, it is assumed that the processing functions of the acquiring function 150a, the display control function 150b, the generating function 150c, and the interface function 150d are implemented by a single processing circuit 150. However, it is also possible to configure the processing circuit 150 as a combination of a plurality of independent processors, and to implement the functions by causing the processors to execute the respective computer programs. In other words, each of the above-mentioned functions may be configured as a computer program, and a single processing circuit 150 may execute corresponding one of such computer programs. As another example, certain functions may be implemented by a dedicated, independent circuit executing a computer program. In FIG. 2, the acquiring function 150a, the display control function 150b, the generating function 150c, and the interface function 150d are examples of an acquiring unit, a display controller, a generating unit, and a controller, respectively. The sequence control circuit 120 is an example of a sequence controller. The display 135 and the gantry panel 170 are examples of the display unit.

The functions of the acquiring function 150a and the display control function 150b will be described later in detail.

The word “processor” used in the above explanation means, for example, a central processing unit (CPU), a graphical processing unit (GPU), an application specific integrated circuit (ASIC), or a programmable logic device (e.g., a simple programmable logic device (SPLD), a complex programmable logic device (CPLD), or a field programmable gate array (FPGA)). The processor implements the functions by reading and executing the computer programs stored in the memory 132.

Instead of storing the computer programs in the memory 132, the computer programs may also be directly incorporated in the processor circuit. In such a configuration, the processor implements their functions by reading and executing the computer programs incorporated in the circuit. In the same manner, circuits such as the couch control circuit 106, the transmitter circuit 108, and the receiver circuit 110 are implemented as electronic circuits such as processors described above.

The processing circuit 150 causes the acquiring function 150a to acquire various types of information of the magnetic resonance imaging system 100. As an example, the processing circuit 150 causes the acquiring function 150a to acquire information of the type of a pulse sequence executed by the sequence control circuit 120, from the sequence control circuit 120. The processing circuit 150 also causes the acquiring function 150a to acquire images via the first camera 140 or the second camera 141. In addition, the processing circuit 150 also causes the acquiring function 150a to acquire the position information of the couchtop 105a from the couch control circuit 106.

The processing circuit 150 causes the display control function 150b to control to capture, to generate, and to display an image by controlling the entire magnetic resonance imaging system 100. For example, the processing circuit 150 causes the display control function 150b to display a first image acquired by the first camera 140 and a second image acquired by the second camera 141, on the display 135. The processing circuit 150 including the display control function 150b also receives an input of imaging conditions (such as imaging parameters) on a GUI, and generates sequence information according to the received imaging conditions. The processing circuit 150 including the display control function 150b also transmits the generated sequence information to the sequence control circuit 120.

The processing circuit 150 causes the generating function 150c to generate an image by applying Fourier transform and other reconstruction processes to k-space data acquired based on magnetic resonance data acquired by causing the sequence control circuit 120 to run a pulse sequence.

The processing circuit 150 causes the interface function 150d to transmit sequence information to the sequence control circuit 120 and to receive magnetic resonance data from the sequence control circuit 120. Upon receiving a piece of magnetic resonance data, the processing circuit 150 including the interface function 150d stores the received magnetic resonance data in the memory 132.

The memory 132 stores therein data such as the magnetic resonance data received by the processing circuit 150 including the interface function 150d, and image data generated by the processing circuit 150 including the generating function 150c. Examples of the memory 132 include a random-access memory (RAM), a semiconductor memory device such as a flash memory, a hard disk, and an optical disc.

The input device 134 receives various instructions and information that are input from an operator. Examples of the input device 134 include a pointing device such as a mouse or a trackball, a selection device such as a mode switch, and an input device such as a keyboard. The display 135 displays, under the control of the processing circuit 150 including the display control function 150b, the first image acquired by the first camera 140, the second image acquired by the second camera 141, a graphical user interface (GUI) for receiving an input of an imaging condition, and an image generated by the processing circuit 150 including the generating function 150c, for example. The display 135 is a display device such as a liquid crystal display.

Background information related to the embodiments will now be explained.

In a medical imaging device such as an MRI device, it is desirable to automate patient settings for a subject.

It is possible to facilitate such patient setting by installing a camera on the ceiling of an imaging room, shooting an image of the subject from a position above the subject, and acquiring information about the subject, for example.

Once the subject is moved to the imaging area, it is also desirable to acquire an image of the subject inside of the bore, in addition to the image shot from above.

In the light of this background, in order to improve user convenience, the display control device 130 according to the embodiment includes the processing circuit 150. The processing circuit 150 causes the acquiring function 150a to acquire the first image shot by the first camera 140 capable of shooting an image of the subject P outside the bore, and the second image shot by the second camera 141 capable of shooting an image of the subject inside the bore. The processing circuit 150 then causes the display control function 150b to display the first image and the second image on the display 135.

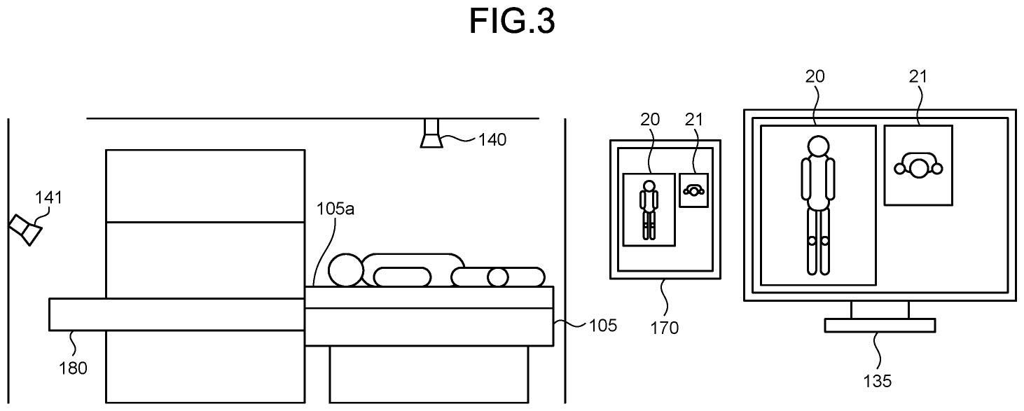

In the first embodiment, a plurality of images from the respective cameras are displayed simultaneously on the gantry panel 170 or the console 135. Such a configuration is illustrated in FIG. 3.

In FIG. 3, the first camera 140 capable of shooting an image of the subject P outside the bore is installed on the ceiling, and the second camera 141 capable of shooting an image of the subject P inside the bore is installed on a side wall. The subject P is laid on the couchtop 105a of the couch 105, and moved along a gantry rail 180. At this time, in the display control device 130 according to the first embodiment, the display control function 150b included in the processing circuit 150 displays a first image 20 shot by the first camera 140 capable of shooting an image of the subject P outside the bore and a second image 21 shot by the second camera 141 capable of shooting an image of the subject inside the bore, simultaneously on the display 135 or the gantry panel 170. In other words, the display control device 130 displays a plurality of images from a plurality of respective cameras simultaneously, on the gantry panel 170 or the display 135.

The second camera 141 may shoot an image of the subject P in real time. In such a case, the processing circuit 150 causes the acquiring function 150a to acquire the second image shot by the second camera 141 in real time. The processing circuit 150 causes the display control function 150b to display the first image 20 of the subject P on the gantry panel 170 or the display 135, and to display the second image 21 shot in real time by the second camera 141 on the gantry panel 170 or the display 135 in real time. At this time, the first image 20 that the processing circuit 150 causes the display control function 150b to display on the gantry panel 170 or the display 135 may be an image not shot in real time, but may be a past image taken before the subject P is moved inside the bore.

In the manner described above, in the first embodiment, the images from the respective cameras are displayed simultaneously on the gantry panel 170 or the console 135. In this manner, it is possible to provide the images to the user effectively, so as to facilitate understanding of the patient's condition, and to improve the user convenience. For example, the position of the subject P can be checked from a plurality of viewpoints using the cameras, so that the user convenience is improved.

### Second Embodiment

In the first embodiment, an example in which a plurality of images from a plurality of cameras are displayed simultaneously on the gantry panel 170 or the console 135. By contrast, in the second embodiment, the image being displayed is switched between the images from the respective cameras. In this manner, the position of the subject P can be checked from the viewpoints of a plurality of cameras, as required, so that the user convenience is improved.

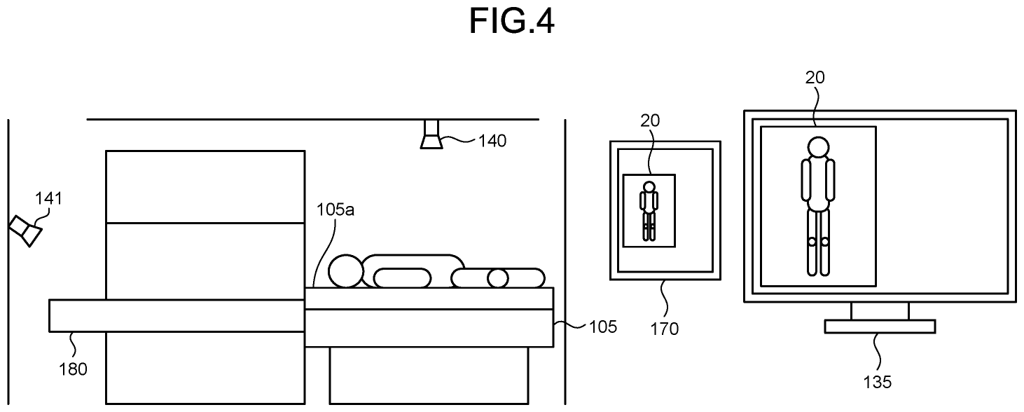

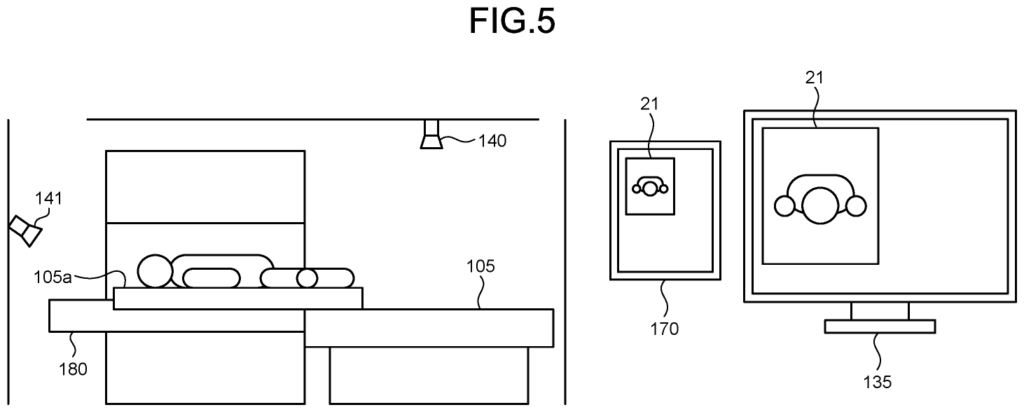

In FIGS. 4 and 5, the first camera 140 capable of shooting an image of the subject P outside the bore is installed on the ceiling, and the second camera 141 capable of shooting an image of the subject P inside the bore is installed on the side wall. The subject P is laid on the couchtop 105a of the couch 105, and moved along the gantry rail 180. FIG. 4 illustrates a configuration in which the subject P is outside the bore, and FIG. 5 illustrates a configuration in which the subject P is inside the bore.

At this time, in the second embodiment, the processing circuit 150 causes the display control function 150b to switch between the first image 20 and the second image 21 as the image being displayed on the display 135 or the gantry panel 170.

For example, as illustrated in FIG. 4, when the subject P is outside the bore, because the subject P is not inserted into the bore and it is sufficient to use only the first camera 140, the processing circuit 150 causes the display control function 150b to display the first image 20 shot by the first camera 140 capable of shooting an image of the subject P outside the bore, on the display 135 or the gantry panel 170.

At the timing at which the couchtop 105a is inserted into the bore, the processing circuit 150 causes the display control function 150b to switch the image being displayed on the display 135 and the gantry panel 170.

For example, when the second camera 141 detects the subject P, the processing circuit 150 causes the display control function 150b to switch the image being displayed on the display 135 or the gantry panel 170 from the first image 20 to the second image 21. For example, the processing circuit 150 causes the acquiring function 150a to acquire the position of the couchtop 105a via the couch control circuit 106 and causes the display control function 150b to switch the image being displayed on the display 135 or the gantry panel 170, from the first image 20 to the second image 21 based on the acquired position.

For example, the processing circuit 150 may also cause the display control function 150b to switch the image being displayed on the display 136 or the gantry panel 170 from the first image 20 to the second image 21 when the subject P is no longer detected by the first camera 140. As an example, the processing circuit 150 causes a determining function, not illustrated, to perform image recognition processing, such as contour extraction, on the first image 20 and to determine whether the subject P is included in the first image 20, and if the subject P is no longer detected by the first camera 140, causes a determining function to switch the image being displayed on the display 136 or the gantry panel 170 from the first image 20 to the second image 21 based on the determination result.

As a result, while the subject P is inside the bore, as illustrated in FIG. 5, the processing circuit 150 causes the display control function 150b to display the second image 21 shot by the second camera 141 capable of shooting an image of the subject in the bore, on the display 135 or the gantry panel 170.

In other words, in the second embodiment, the processing circuit 150 causes the display control function 150b to switch between the first camera 140 capable of shooting an image of the subject P outside the bore and the second camera 141 capable of shooting an image of the subject inside the bore, as appropriate. In this manner, by allowing the display unit to display a required camera depending on the situation, it allows a user to understand the condition of the patient more accurately, the convenience for the user is improved.

### Third Embodiment

Explained in the second embodiment is an example in which the image being displayed on the display unit is switched depending on whether the subject P is inside or outside the bore. In a third embodiment, the image being displayed on the display unit is switched based on the body posture of the subject P. For example, in the third embodiment, depending on whether the subject P is moved into the bore head-first or feet-first, the processing circuit 150 causes the display control function 150b to switch the image being displayed on the display 135 or the gantry panel 170.

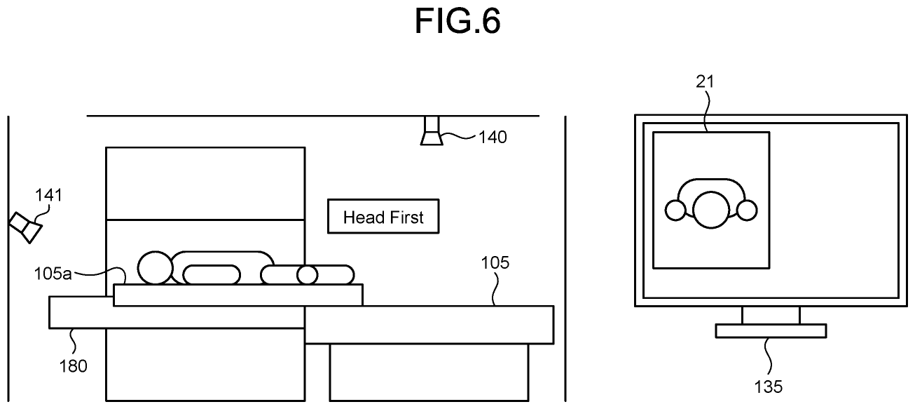

For example, as illustrated in FIG. 6, when the subject P is moved into the bore from his/her head (head-first), information of the second image 21 acquired by the second camera 141 capable of shooting an image inside the bore is relatively important. Therefore, the processing circuit 150 causes the display control function 150b to display the second image 21 acquired by the second camera 141 capable of shooting an image inside the bore, on the display 135 or the gantry panel 170.

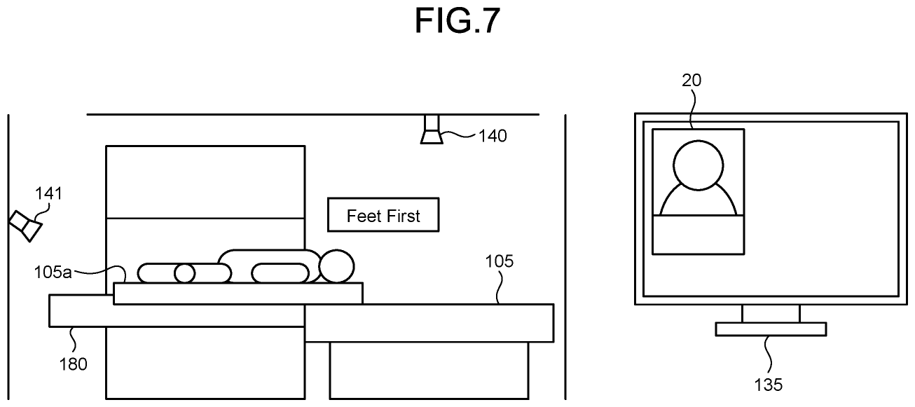

By contrast, when the subject P is moved into the bore from his/her feet (feet first), as illustrated in FIG. 7, an image of the subject P may be shot with his/her head outside the bore, for example. In such a case, it may be desirable to display information of the first image 20 acquired by the first camera 140 capable of shooting an image outside the bore, for a purpose such as checking the facial expression of the subject P. Therefore, in the feet-first situation, the processing circuit 150 causes the display control function 150b to display the first image 20 shot by the first camera 140 capable of shooting an image outside the bore, on the display 135 or the gantry panel 170.

In such a case, as illustrated in FIG. 7, the processing circuit 150 may cause the display control function 150b to remove the unnecessary parts that are not related to the subject P from the image shot by the first camera 140, and display the needed part that is enlarged as the first image 20 on the display 135 or the gantry panel.

The processing circuit 150 may also cause the display control function 150b to display the second image 21 acquired by the second camera 141 capable of shooting an image inside of the bore, on the display 135 or the gantry panel 170, when requested by the user. As an example, when the processing circuit 150 receives a command for displaying the second image 21 because the user selected a button displayed on the display unit with the input device 134, the processing circuit 150 may cause the display control function 150b to display the second image 21, in addition to the first image 20, on the display 135 or the gantry panel 170.

In the third embodiment described above, depending on the body posture of the subject P, the processing circuit 150 causes the display control function 150b to switch the image being displayed on the display 135 or the gantry panel 170. In this manner, an image corresponding to the type of imaging is displayed automatically, and the need for the user to switch the two images is eliminated, so that the examination throughput and the user convenience are improved.

According to at least one of the embodiments described above, it is possible to improve the user convenience.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A display control device comprising:
a processing circuit configured to acquire a first image shot by a first camera capable of shooting an image of a subject outside a bore and a second image shot by a second camera capable of shooting an image of the subject inside the bore, and to display the first image and the second image on a display.

2. The display control device according to claim 1, wherein the processing circuit is configured to display the first image and the second image simultaneously on the display.

3. The display control device according to claim 1, wherein the processing circuit is configured to acquire the second image in real time, and to display the first image of the subject who is laid on a couchtop outside the bore, on the display, and to display the second image in real time on the display.

4. The display control device according to claim 1, wherein the processing circuit is configured to display the first image and the second image on the display, by switching between the first image and the second image.

5. The display control device according to claim 4, wherein the processing circuit is configured to switch between the first image and the second image at a timing at which a couchtop is inserted into the bore.

6. The display control device according to claim 4, wherein the processing circuit is configured to switch the image being displayed on the display from the first image to the second image when the second camera detects the subject.

7. The display control device according to claim 4, wherein the processing circuit is configured to switch the image being displayed on the display from the first image to the second image when the subject is no longer detected by the first camera.

8. The display control device according to claim 4, wherein the processing circuit is configured to switch the first image and the second image, as an image being displayed, depending on a body posture of the subject.

9. The display control device according to claim 1, wherein the processing circuit is configured to display the first image on the display when the subject is moved into the bore head-first, and to display the second image on the display when the subject is moved into the bore feet-first.
